(19) United States
(12) Patent Application Publication
(10) Pub. No.: US 2013/0102624 A1
(43) Pub. Date: Apr. 25, 2013

(54) EARLY DETECTION OF THIAMINE DEFICIENCY

(71) Applicant: John V. Schloss, Portland, ME (US)

(72) Inventor: John V. Schloss, Portland, ME (US)

(21) Appl. No.: 13/475,260

(22) Filed: May 18, 2012

Related U.S. Application Data
(60) Provisional application No. 61/488,027, filed on May 19, 2011.

(51) Int. Cl. H01J 49/00

(52) U.S. Cl. CPC H01J 49/0027
USPC 514/276; 250/282

(57) ABSTRACT

A method is provided for determining the thiamine status of a human or animal based on the relative levels of thiamine and its metabolites. Certain embodiments of the present invention also provide methods for determining the effectiveness of a thiamine deficiency treatment.

### CROSS-REFERENCE TO RELATED APPLICATION(S)

This patent application claims the benefit of priority of U.S. application Ser. No. 61/488,027, filed May 19, 2011, which application is herein incorporated by reference.

### FUNDING

The invention described herein was made with support from the Gustavus and Louise Pfeiffer Research Foundation. Royalty free use for non-commercial projects will be permitted, as a condition of support from the Gustavus and Louise Pfeiffer Research Foundation.

### BACKGROUND OF THE INVENTION

Thiamine deficiency can lead to irreversible neurological damage and death. It is estimated that approximately 28% of the U.S. population suffers from chronic thiamine deficiency, without any clear outward clinical manifestations. Thiamine deficiency results from consumption of ‘empty calories’, foods that have little or no thiamine content; consumption of foods containing enzymes that can degrade thiamine during the digestion process, e.g., thiaminases; and the presence of bacteria in the intestinal flora that can degrade thiamine prior to absorption from the digestive tract. Individuals who are subject to a thiamine-deficient diet; consume foods containing enzymes that degrade thiamine; or harbor bacteria in their intestinal flora that degrade thiamine are unlikely to be aware of this, even after the clinical symptoms of thiamine deficiency become manifest. As such, there is a need for a rapid and noninvasive method to identify individuals who are subject to thiamine deficiency.

### SUMMARY OF THE INVENTION

As described herein, certain embodiments of the present invention provide a method for the early detection of thiamine deficiency.

Certain embodiments of the present invention provide methods for identifying an animal that has thiamine deficiency, comprising determining the ratio of 4-methyl-5-(2-hydroxyethyl)-thiazole (TMA) to 2-methyl-4-amino-5-hydroxymethylpyrimidine (PMA) in a biological sample from the animal, wherein a ratio of greater than about five indicates the animal has thiamine deficiency.

Certain embodiments of the present invention provide methods for determining the effectiveness of a thiamine deficiency treatment, comprising determining the ratio of 4-methyl-5-(2-hydroxyethyl)-thiazole (TMA) to 2-methyl-4-amino-5-hydroxymethylpyrimidine (PMA) in a first biological sample taken from an animal before thiamine deficiency treatment and in a second biological sample taken from the animal after the thiamine deficiency treatment, wherein the thiamine deficiency treatment is effective if the ratio of TMA to PMA in the second sample is less than the ratio of TMA to PMA in the first sample.

### BRIEF DESCRIPTION OF THE FIGURES

FIG. 1. Analysis of a mixture of thiamine derivatives by liquid chromatography/tandem mass spectrometry (LC-MS/MS).

FIG. 2. The rate of excretion of thiamine in urine is illustrated.

FIG. 3. The rate of excretion of TMA and PMA in urine is shown.

FIG. 4. The ratio of thiazole and pyrimidine metabolites as a function of dietary status.

### DETAILED DESCRIPTION

The present invention demonstrates that the ratio of two different thiamine metabolites in urine, TMA (4-methyl-5-(2-hydroxyethyl)-thiazole or thiazole metabolite A) and PMA (2-methyl-4-amino-5-hydroxymethylpyrimidine or pyrimidine metabolite A), is a sensitive measure of thiamine deficiency. The development of such an assay will be valuable in assessing thiamine deficiency and response to treatment.

An individual's thiamine status results from a balance of uptake of dietary thiamine; thiamine degradation during metabolism of carbohydrates, proteins, and fats; and thiamine degradation by thiaminase I and II present in foods or produced by intestinal microbes. The dietary requirement for thiamine or vitamin B1 depends on total calories consumed. Research suggests that oxidative degradation of thiamine is a contributing factor in the linkage between the dietary requirement for thiamine and total caloric intake (Bunik, et al., (2007) Neurochem Res 32:871-891; Bunik, et al., (2011) Adv. Enzymol Relat Areas Mol Biol 77:307-360).

Based on lymphocyte proliferation assays, it has been estimated that up to 28% of the general population suffers from thiamine deficiency (Bucci LR (1994) Am Clin Lab 13:10-11). Thiamine deficiency is a likely determinant of individual susceptibility to noise-induced hearing loss and related pathology (e.g., tinnitus), and other pathologies linked to glutamate receptor dysfunction, such as: blood disorders, diabetes, heart disease, osteoporosis, atherosclerosis, and neurological disorders, such as Wernicke-Korsakoff syndrome (Lonsdale D (2006) Evid Based Complement Alternat Med 3:49-59; Todd K G, Butterworth R F (1998) Exp Neurol 149:130-8; Nicoletti F, et al. (2007) Psychoneuroendocrinology 32:S40-5). Defects in the high-affinity thiamine transporters (Thiamine-Responsive Megaloblastic Anemia (IRMA) or Rogers' syndrome) result in anemia, diabetes, and hearing loss (Porter F S, et al. (1969) J Pediatr 74:494-504; Oishi K, et al. (2002) Hum Mol Genet. 11:2951-60; Liberman M C, et al. (2006) JARO 7: 211-217).

Thiamine deficiency in the general population is a consequence of consumption of ‘empty calories’ (e.g., alcoholism, polished rice, or sugar-containing soft drinks) (Lonsdale D (2006) Evid Based Complement Alternat Med 3:49-59); chemicals in the diet or pharmaceuticals that block conversion of thiamine to its active form (e.g., pyrithiamine or metronidazole) (Todd K G, Butterworth R F (1998) Exp Neurol 149:130-8; Alston T A, Abeles R H (1987) Arch Biochem Biophys 257:357-62); thiamine degrading enzymes in the diet (e.g., thiaminases present in animals or plants consumed) (Lonsdale D (2006) Evid Based Complement Alternat Med 3:49-59; Adamolekun B, Ibikunie F R (1994) Acta Neurol Scand 90:309-11); microorganisms in the gut that degrade dietary thiamine (e.g., Bacillus thiaminolyticus) (Nakamura L K (1990) Int J Syst Bacteriol 40:242-6; Edwin E E, et al. (1978) J Appl Microbiol 44:305-12; Princewill T J T (1980) J Appl Bacteriol 48:249-52); and various disease states (e.g., congestive heart failure, infections, or gastroenteritis) (Ouytang J, et al. (2008) Ann Clin Lab Sci 38:393-400; Hanninen S A, et al. (2006) JACC 47:354-361; Araya M, et al. (1975) Aust N Z J Med 5, 239-50). Detection of thiamine deficiency usually follows clinical manifestation of symptoms associated with its deficiency, such as: mental confusion, motor ataxia, peripheral neuropathy, or cardiomegaly (Lonsdale D (2006) Evid Based Complement Alternat Med 3:49-59). Unfortunately, not all of the pathology associated with thiamine deficiency is reversible by dietary thiamine supplementation. Accordingly, there is a need for a rapid noninvasive method to diagnose thiamine deficiency and facilitate detection of deficiency at an early point before clinical symptoms appear.

The common methods currently in use for diagnosis of thiamine deficiency include: functional erythrocyte transketolase activation by exogenous thiamine pyrophosphate (TPP) (Finglas P M (1993) Int J Vitam Nutr Res 63:270-274); the phytohemagglutinin (PHA)-stimulated response of cultured lymphocytes to thiamine-deficient media (Bucci L R (1994) Am Clin Lab 13:10-11; Shive W, et al. (1986) Proc Natl Acad Sci USA 83:9-13); and the concentration of TPP in erythrocytes (Bettendorff L, et al. (1986) J Chromat Biomed Appl 382:297-302; Floridi A, et al. (1984) Int J Vitam Nutr Res 54:165-171). Unlike the methods described herein, where there is a >150-fold increase in the ratio of TMA/PMA in the early stages of thiamine deficiency (see FIG. 4), the lymphocyte proliferation assay (2-fold difference); erythrocyte transketolase stimulation by TPP (25% increase); level of thiamine excreted (3-fold difference); return of a 1-5 mg dose of thiamine (load test, 4-fold difference) and TPP levels in erythrocytes (4-fold difference) only change by modest amounts during advanced thiamine deficiency, when clinical symptoms are already manifest (Shive et al., (1986) Proc Natl Acad Sci USA 83:9-13; Sauberlich H E (1984) Annu Rev Nutr 4:377-407; Vuilleumier, et al., (1983) Int J Vitam Nutr Res 53:359-370; Bettendorff et al., (1986) J Chromat Biomed Appl 382:297-302; Floridi et al., (1984) Int J Vitam Nutr Res 54:165-171). Additionally, all of these previous methods are invasive in that they require collection of blood samples and require time-consuming analytical methodology to obtain results.

The present invention describes a rapid, non-invasive method that can identify thiamine deficiency at an early stage, before clinical symptoms are manifest. As demonstrated by the Examples below, the ratio of two metabolites, derived from different portions of the thiamine molecule, can be used to identify a diet that is leading to thiamine deficiency. These two metabolites are TMA (4-methyl-5-(2-hydroxyethyl)-thiazole) and PMA (2-methyl-4-amino-5-hydroxymethylpyrimidine). During periods of fasting, the levels of thiamine, TMA, and PMA in urine are reduced. This indicates that thiamine is retained by a fasting individual. However, when empty calories are consumed, the urinary levels of thiamine and these two metabolites are elevated. In particular, the ratio of the thiazole and pyrimidine metabolites can increase by more than 100-fold. Consumption of thiamine after a period of induced deficiency results in a transient reduction of this ratio to less than 20% of its typical fasting value. Accordingly, the ratio of TMA to PMA can be used to identify a thiamine deficiency.

Additionally, urinary thiamine metabolites have substantial potential as biomarkers of thiaminases produced by micro flora in the gut and the effect of antibiotics on intestinal microbes; genetic disorders; and thiamine deficiency states. The degradation products of thiamine can be used to monitor its use in carbohydrate, protein, and fat metabolism; changes in metabolism involving different thiamine-dependent enzymes may result in different patterns of thiamine degradation products in urine. Thus, urinary thiamine metabolites may also be useful to: (1) identify genetic disorders involving elevated levels of thiamine cofactor oxidation; (2) identify individuals at high risk for thiamine deficiency due to dietary thiaminases; (3) identify individuals harboring intestinal organisms that produce thiaminases; and (4) monitor the elimination of thiaminase-producing microbes by use of antibiotics. The TMA/PMA ratio may also be useful as a newborn infant screen for thiamine-responsive megaloblastic anemia syndrome, a rare disorder involving a defect in high-affinity thiamine transporters. Additionally, this assay may also be used to monitor the thiamine status of patients with congestive heart failure, as these patients have a higher incidence of thiamine deficiency.

As described herein, certain embodiments of the present invention provide methods for identifying an animal that has a thiamine deficiency, comprising determining the level of thiamine, one or more thiamine metabolites, or thiamine and one or more thiamine metabolites in a biological sample from the animal, wherein the levels are an indication of thiamine deficiency. In certain embodiments, the level of thiamine and the levels of one or more thiamine metabolites are compared, wherein the relative levels are an indication of thiamine deficiency. In certain embodiments, the levels of thiamine metabolites are compared, wherein the relative levels are an indication of thiamine deficiency.

In certain embodiments, the thiamine metabolites are 2-methyl-4-amino-5-hydroxymethylpyrimidine (PMA), 4-methyl-5-(2-hydroxyethyl)-thiazole (TMA), thiamine disulfide, thiamine thiazolone, 2-methyl-4-amino-5-pyrimidinecarboxylic acid (PMB), 2-methyl-4-hydroxy-5-pyrimidinecarboxylic acid (PMC), 4-methyl-5-thiazolecarboxylic acid (TMB), 2-oxo-4-methyl-5-(2-hydroxyethyl)-thiazole (TMC), or 2-oxo-4-methyl-5-thiazolecarboxylic acid (TMD).

Certain embodiments of the present invention provide methods for identifying an animal that has thiamine deficiency, comprising determining the ratio of 4-methyl-5-(2-hydroxyethyl)-thiazole (TMA) to 2-methyl-4-amino-5-hydroxymethylpyrimidine (PMA) in a biological sample from the animal, wherein a ratio of greater than about five indicates the animal has thiamine deficiency.

In certain embodiments of the invention, the methods further comprise administering to the animal with thiamine deficiency a therapy effective to reduce the thiamine deficiency. In certain embodiments of the invention, the therapy is effective to decrease the ratio of 4-methyl-5-(2-hydroxyethyl)-thiazole (TMA) to 2-methyl-4-amino-5-hydroxymethylpyrimidine (PMA). In certain embodiments of the invention, the ratio is reduced to less than about one.

In certain embodiments, the therapy effective to reduce the thiamine deficiency is thiamine (also known as Aneurin), a salt thereof, or a thiamine precursor. As described herein, a thiamine precursor is something that will produce thiamine in an animal. In certain embodiments, the thiamine precursor is selected from, thiamine disulfide, thiamine disulfide butyrate, allithiamine (thiamine allyl disulfide), S-benzoylthiamine, Benfotiamine (S-benzoylthiamine O-monophosphate), Sulbutiamine (Arcalion, the O-bis-2-methylpropanoate ester of thiamine disulfide), Prosultiamine (thiamine propyl disulfide, also known as Alinamin, Binova, Jubedel, Taketron, Thiobeta, and Thiotiamina), Fursulthiamine (thiamine tetrahydrofurfuryl disulfide, also known as Adventan, Alinamin-F, Benlipoid, Bevitol, Lipophil, and Judolor), thiamine monophosphate, thiamine diphosphate (also known as thiamine pyrophosphate, ThDP, or TPP), thiamine triphosphate (ThTP or TTP), adenosine thiamine triphosphate (AThTP), and adenosine thiamine diphosphate (AThDP). In certain embodiments the thiamine salt is thiamine hydrochloride.

In certain embodiments greater than about 1 mg of thiamine hydrochloride is administered to the animal.

Certain embodiments of the present invention provide methods for determining the effectiveness of a thiamine deficiency treatment, comprising determining the ratio of 4-methyl-5-(2-hydroxyethyl)-thiazole (TMA) to 2-methyl-4-amino-5-hydroxymethylpyrimidine (PMA) in a first biological sample taken from an animal before thiamine deficiency treatment and in a second biological sample taken from the animal after the thiamine deficiency treatment, wherein the thiamine deficiency treatment is effective if the ratio of TMA to PMA in the second sample is less than the ratio of TMA to PMA in the first sample.

In certain embodiments of the invention the ratio of TMA to PMA in the second sample is less than about half of the ratio of TMA to PMA in the first sample. In certain embodiments the ratio of TMA to PMA in the second sample is less than about one fifth of the ratio of TMA to PMA in the first sample. In certain embodiments the ratio of TMA to PMA in the second sample is less than about one tenth of the ratio of TMA to PMA in the first sample. In certain embodiments, the ratio of TMA to PMA in the second sample is less than about five and greater than about one; and wherein the ratio of TMA to PMA in the first sample is greater than about five. In certain embodiments, the ratio of TMA to PMA in the second sample is less than about five and greater than about one tenth; and wherein the ratio of TMA to PMA in the first sample is greater than about five. In certain embodiments the ratio of TMA to PMA in the second sample is less than about one; and wherein the ratio of TMA to PMA in the first sample is greater than about five.

In certain embodiments the second sample was taken from the animal from between about 10 hours to about 30 hours after thiamine deficiency treatment.

As used herein, the terms “treat” and “treatment” can refer to therapeutic treatment. In some embodiments of the invention, the object is to reduce thiamine deficiency. Certain embodiments of the invention relate to determining the effectiveness of a thiamine deficiency treatment.

In certain embodiments of the invention, the thiamine deficiency treatment comprises administering to the animal thiamine, a salt thereof, or a thiamine precursor. In certain embodiments, the thiamine precursor is selected from thiamine disulfide, thiamine disulfide butyrate, allithiamine (thiamine allyl disulfide), S-benzoylthiamine, Benfotiamine (S-benzoylthiamine O-monophosphate), Sulbutiamine (Arcalion, the O-bis-2-methylpropanoate ester of thiamine disulfide), Prosultiamine (thiamine propyl disulfide, also known as Alinamin, Binova, Jubedel, Taketron, Thiobeta, and Thiotiamina), Fursulthiamine (thiamine tetrahydrofurfuryl disulfide, also known as Adventan, Alinamin-F, Benlipoid, Bevitol, Lipophil, and Judolor), thiamine monophosphate, thiamine diphosphate (also known as thiamine pyrophosphate, ThDP, or TPP), thiamine triphosphate (ThTP or TTP), adenosine thiamine triphosphate (AThTP), and adenosine thiamine diphosphate (AThDP). In certain embodiments the thiamine salt is thiamine hydrochloride. In certain embodiments greater than about 1 mg of thiamine hydrochloride is administered to the animal.

In certain embodiments of the invention, the ratio(s) is determined by analyzing the sample by liquid chromatography/tandem mass spectrometry (LC-MS/MS), as described herein.

In certain embodiments the invention provides a method to treat thiamine deficiency in an animal comprising administering thiamine, a salt thereof, or a thiamine precursor to the animal until the ratio of 4-methyl-5-(2-hydroxyethyl)-thiazole (TMA) to 2-methyl-4-amino-5-hydroxymethylpyrimidine (PMA) in a biological sample from the animal is less than five.

In certain embodiments the invention provides a method comprising administering thiamine, a salt thereof, or a thiamine precursor to an animal until the ratio of 4-methyl-5-(2-hydroxyethyl)-thiazole (TMA) to 2-methyl-4-amino-5-hydroxymethylpyrimidine (PMA) in a biological sample from the animal is less than about one.

In certain embodiments the invention provides a method for treating a subject having a thiamine deficiency, comprising administering an amount of thiamine, a salt thereof, or a thiamine precursor to the subject, wherein the subject has been identified as being at risk for having a thiamine deficiency, wherein the ratio of 4-methyl-5-(2-hydroxyethyl)-thiazole (TMA) to 2-methyl-4-amino-5-hydroxymethylpyrimidine (PMA) in a urine sample from the subject has been determined to be greater than about five, wherein the amount of thiamine, the salt thereof, or the thiamine precursor is effective to decrease the ratio of TMA to PMA in a urine sample from the subject to less than about one.

In certain embodiments the invention provides a method for treating thiamine deficiency in an animal in need of such therapy comprising: I) administering thiamine, a salt thereof, or a thiamine precursor to the animal, 2) measuring the ratio of 4-methyl-5-(2-hydroxyethyl)-thiazole (TMA) to 2-methyl-4-amino-5-hydroxymethylpyrimidine (PMA) in a biological sample from the animal within 72 hours of administration, and 3) administering additional thiamine, a salt thereof, or a thiamine precursor to the animal if the ratio is greater than 5.

In certain embodiments the invention provides a method comprising treating thiamine deficiency in a human that is aware of having a ratio of 4-methyl-5-(2-hydroxyethyl)-thiazole (TMA) to 2-methyl-4-amino-5-hydroxymethylpyrimidine (PMA) of greater than 5 and that is therefore aware of being in need of such therapy comprising administering thiamine, a salt thereof, or a thiamine precursor to the human.

In certain embodiments the invention provides a method for detecting the presence of a biomarker in an animal comprising identifying a ratio of 4-methyl-5-(2-hydroxyethyl)-thiazole (TMA) to 2-methyl-4-amino-5-hydroxymethylpyrimidine (PMA) of greater than about 5 in a sample from the animal.

As described herein, a sample may be any material from the animal that can be used to determine the ratio of 4-methyl-5-(2-hydroxyethyl)-thiazole (TMA) to 2-methyl-4-amino-5-hydroxymethylpyrimidine (PMA). In certain embodiments the sample is a liquid, tissue or bone from the animal. In certain embodiments the sample is selected from urine, blood, tissue, sweat, tears, saliva, cerebrospinal fluid, intraperitoneal fluid, vitreous humor, skin, bone, bone marrow, pancreas, spleen, spinal cord, brain, dura mater, heart, lung, kidney, endocrine glands, bladder, blood vessel, testicle, ovary, uterus, umbilical cord, placenta, muscle, pancreas, spleen, cartilage, connective tissue, intestine, colon, stomach, esophagus, tongue, cochlea, cochlear fluid, inner ear, gall bladder, and liver biopsy. In certain embodiments the sample is urine. It is understood that certain types of samples may be processed (e.g., diluted, subjected to sonic or mechanical disruption or otherwise dispersed into aqueous or organic media, passed through a size-exclusion membrane, treated with an organic solvent such as methanol, or subjected to solid phase extraction) prior to analysis (e.g. LC-MS/MS).

In certain embodiments, the animal is a mammal, fish, bird, reptile, amphibian or invertebrate animal. In certain embodiments the animal is a human, cow, bull, horse, sheep, pig, goat, dog, cat, pig, rabbit, ox, mouse, rat, chinchilla, chimpanzee, gorilla, ape, monkey, bear, wolf, zebra, lion, tiger, llama, seal, ocelot, whale, porpoise, deer, moose, parrot, falcon, eagle, chicken, duck, turtle, alligator, crocodile, monitor, frog, toad, salmon, tuna, tilapia, cod, salmon, catfish, trout, herring, whiting, flounder, perch, plaice, sprat, vendace, whitefish, pike, mackerel, saithe, pout, carp, charr, grayling, halibut, haddock, shellfish, lobster, shrimp, squid, octopus. In certain embodiments the animal is a human.

Certain embodiments of the invention will now be illustrated by the following non-limiting Examples.

### Example 1

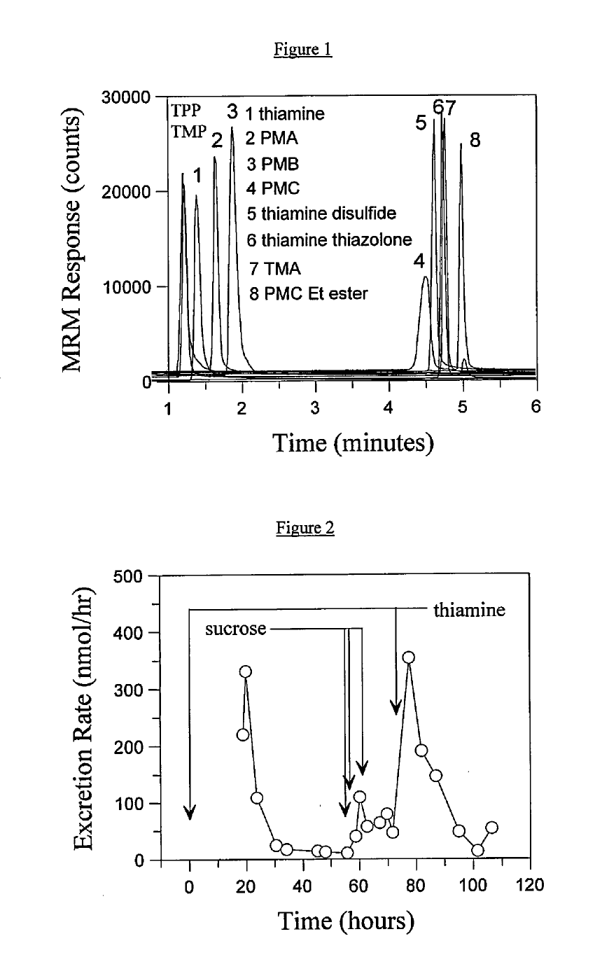

A method to analyze thiamine and thiamine derivatives is described herein. FIG. 1 illustrates a six-minute chromatographic separation of thiamine pyrophosphate (TPP), thiamine monophosphate (TMP), thiamine (1), PMA (2) (2-methyl-4-amino-5-hydroxymethylpyrimidine), PMB (3) (2-methyl-4-amino-5-pyrimidinecarboxylic acid), PMC (4) (2-methyl-4-hydroxy-5-pyrimidinecarboxylic acid), thiamine disulfide (5), thiamine thiazolone (6), TMA (7) (4-methyl-5-(2-hydroxyethyl)-thiazole), and PMC Et (8) (the ethyl ester of PMC). Detection of all compounds as they elute from an Agilent 1200 series chromatograph was accomplished by multiple reaction monitoring with an Agilent 6460 LC-MS/MS. The chromatographic column employed was a Polaris 5 C18-A, 150×2 mm, obtained from Agilent. After injecting 10 microliters (μL) of a mixture of thiamine and thiamine derivatives, the column was eluted for 2 minutes with 10 millimolar (mM) ammonium formate, pH 3, dissolved in water at 0.5 milliliters/minute (mL/min). This was followed by eluting the column with a linear gradient composed of 10 mM ammonium formate, pH 3, (buffer A) and 0.1% formic acid dissolved in acetonitrile (buffer B). The gradient started with a mixture of 5% buffer A and 95% buffer B and increased to 50% buffer A and 50% buffer B over a four (4) minute period at 35° C. The parent mass for each compound was sequentially selected; subjected to collisional fragmentation; and at least two unique fragments for each compound used for quantitative measurement and qualitative confirmation of structure. The parent or precursor mass (Prec, mass-to-charge ratio or m/z), product ions (Prod qual=product ion used for qualitative confirmation of identity, m/z; Prod quant=product ion used for quantitation, m/z), collisional energy (CE in volts), and fragmentation voltage (Frag) used for different compounds were: thiamine (Prec 265, Prod qual 144, Frag 80, CE 5; Prod quant 122, CE 9), thiamine disulfide (Prec 564.2, Prod qual 123.7, Frag 155, CE 37; Prod quant 110.1, CE 33), TPP (Prec 425.1, Prod qual 303, Frag100, CE 9; Prod quant 122, CE 9), TMP (Prec 345, Prod qual 223.8, Frag 100, CE 9; Prod quant 121.9, CE 15), thiamine thiazolone (Prec 281.1, Prod qual 165.2, Frag 118, CE 17; Prod quant 123.1, CE 21), PMC Et (Prec 183, Prod qual 95.9, Frag 80, CE 13; Prod qual 68.9, CE 21; Prod quant 136.9, CE 9), PMC (Prec 155.1, Prod qual 68.9, Frag 85, CE 17; Prod quant 136.9, CE 5), PMB (Prec 154.1, Prod qual 136.1, Frag 100, CE 13; Prod quant 95, CE 17), TMA (Prec 144, Prod qual 126, Frag 100, CE 15; Prod quant 113.1, CE 20), and PMA (Prec 140.2, Prod qual 122.2, Frag 100, CE 17; Prod quant 81.1, CE 5). In all cases, the resolution of MS1 and MS2 was set to unit resolution; the dwell time was 20; and the polarity was set to positive. Other parameters that were used include: gas temperature=300° C.; gas flow=5 liters/min; nebulizer=45 psi; sheath gas temperature=400° C.; sheath gas flow=11 liters/min; capillary=3500 volts; and nozzle=500 volts. This method was carried out on an Agilent 6460 LC-MS/MS and allowed for rapid determination (6 minutes) of thiamine and its metabolites to concentrations below 1 nM (<0.3 μg/L). For urine samples, assays could be carried out directly without any sample preparation required. Similarly, for tissue extracts, simple dilution of samples prior to analysis by LC-MS/MS was sufficient.

### Example 2

Use of the method described in Example 1 to determine human urinary excretion of thiamine resulted in the data shown in FIG. 2. To obtain a reference value for thiamine excretion rates under fasting conditions, a 100-milligram (mg) sample of thiamine was consumed by a human subject prior to initiating a 56-hour fast. Throughout the experiment, each urine sample was collected and stored at 5° C. after recording the collection time and sample volume. At 56 hours into the fast, 46 grams of sucrose (20 sugar cubes) were consumed as ‘empty calories’ (calories containing no thiamine). Additional sucrose (46 grams) was consumed at 57 and 60 hours after initiation of the fast. The total amount of sucrose consumed was 138 grams (530 kilocalories (kcal)), which is approximately equal to the amount of sugar consumed by the human brain on a daily basis (120 grams of glucose). After a short period following sucrose consumption, 100 mg of thiamine was consumed to reestablish thiamine balance. Peak levels of thiamine excretion following consumption of thiamine before or after the fast were 330 nanomoles/hour (nmol/hr) (pre-fast) and 350 nmol/hr (post-fast; post ‘empty calories’). Only 1.4% of the second sample of thiamine consumed was recovered in the urine (4.1 micromoles (μmol) of the 300 μmol consumed). The small amount of thiamine recovered in the urine is expected due to a combination of the poor oral bioavailability of thiamine and non-renal routes of thiamine clearance (Weber W, et al. (1990) J Pharmacokinet Biopharm 18:501-23; Lonsdale D, et al. (2002) Neuroendocrinology Letters 23:303-308). As the fast progressed, the amount of thiamine excreted in the urine decreased to a low of 10 nmol/hr (80 micrograms/day (μg/day)), indicating that fasting induces conservation of thiamine stores in the body. However, when ‘empty calories’ were consumed, the rate of thiamine excretion dramatically increased to a peak rate of 110 nmol/hr (880 μg/day). These results suggest that the level of thiamine in the diet does not control thiamine excretion rates. The excretion rates presented here, including the increase in thiamine excretion rate concomitant with a thiamine-free diet, are consistent with values reported in earlier studies by use of other methods (Schultz A L, Natelson S (1972) Microchemical Journal 17:109-118; Ziporin Z Z, et al. (1965) J Nutrition 85:287-296).

### Example 3

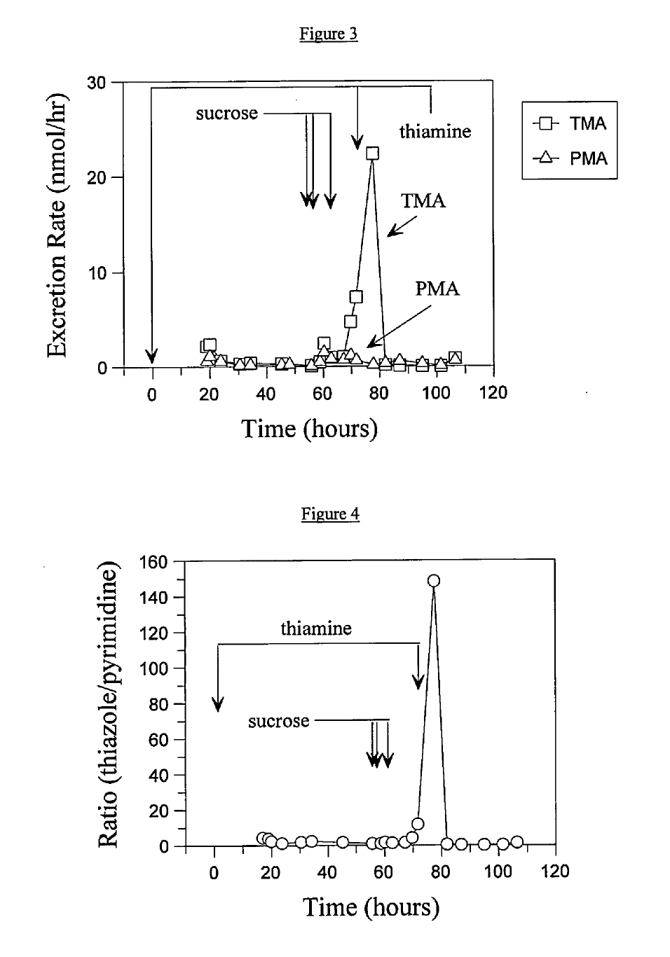

The excretion levels of 4-methyl-5-(2-hydroxyethyl)-thiazole (thiazole metabolite A or TMA) and 2-methyl-4-amino-5-hydroxymethylpyrimidine (pyrimidine metabolite A or PMA) in urine from the same subject are presented in FIG. 3. During fasting both TMA and PMA excretion rates decrease, reaching 0.06 nmol/hr and 0.07 nmol/hr at their lowest point, respectively. Once thiamine-free nutrition (sucrose) is consumed, both metabolites initially increase to about the same excretion rates, similar to the increase observed in thiamine excretion rate. However, after a lag of about 10 hours, there is a dramatic increase in the rate of excretion of TMA. After consumption of thiamine, the excretion rate of both PMA and TMA decrease, but TMA falls to a much greater extent. Before being affected by consumption of thiamine, following consumption of sucrose, the ratio of TMA to PMA (TMA/PMA) is about 150. Following consumption of thiamine, the ratio of TMA to PMA (TMA/PMA) falls to 0.12, which is lower than in the fasting period before consuming sucrose. The change in the ratio of TMA to PMA, as a function of nutritional status, is illustrated in FIG. 4. Unlike the absolute concentrations of thiamine or either metabolite, the ratio of these two metabolites is diagnostic for dietary thiamine restriction. Further, the response of the TMA/PMA ratio to thiamine supplementation depends on thiamine status at the time of supplementation. Use of the ratio of thiazole and pyrimidine metabolites (TMA/PMA) to detect thiamine deficiency has the advantage of being a relative measure of the amounts of two metabolites in the same sample. It does not depend on the absolute concentration of either metabolite. Together with the relative concentration of thiamine in the urine specimen, a single analysis provides a substantial amount of information. When combined with the changes in the concentrations of thiamine, TMA, and PMA, following thiamine consumption, this assay is a powerful indicator of thiamine status.

All documents cited herein are incorporated by reference. While certain embodiments of invention are described, and many details have been set forth for purposes of illustration, certain of the details can be varied without departing from the basic principles of the invention.

The use of the terms “a” and “an” and “the” and similar terms in the context of describing embodiments of invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. In addition to the order detailed herein, the methods described herein can be performed in any suitable order unless otherwise indicated herein or otherwise clearly contradicted by context. The use of any and all examples, or exemplary language (e.g., “such as”) provided herein, is intended merely to better illuminate embodiments of invention and does not necessarily impose a limitation on the scope of the invention unless otherwise specifically recited in the claims. No language in the specification should be construed as indicating that any non-claimed element is essential to the practice of the invention.

1. A method for identifying an animal that has thiamine deficiency, comprising determining the ratio of 4-methyl-5-(2-hydroxyethyl)-thiazole (TMA) to 2-methyl-4-amino-5-hydroxymethylpyrimidine (PMA) in a biological sample from the animal, wherein a ratio of greater than about five indicates the animal has thiamine deficiency.

2. The method of claim 1, wherein the ratio is determined by analyzing the sample by liquid chromatography/tandem mass spectrometry (LC-MS/MS).

3. (canceled)

4. The method of claim 2, wherein the sample is urine.

5. (canceled)

6. (canceled)

7. The method of claim 1, wherein the animal is a human.

8. The method of claim 1, further comprising administering to the animal with thiamine deficiency a therapy effective to reduce the thiamine deficiency.

9. The method of claim 8, wherein the therapy is effective to decrease the ratio of 4-methyl-5-(2-hydroxyethyl)-thiazole (TMA) to 2-methyl-4-amino-5-hydroxymethylpyrimidine (PMA).

10. The method of claim 9, wherein the ratio is reduced to less than about one.

11. The method of claim 8, wherein the therapy effective to reduce the thiamine deficiency is thiamine, a salt thereof, or a thiamine precursor.

12. (canceled)

13. The method of claim 11 wherein the thiamine salt is thiamine hydrochloride.

14. (canceled)

15. A method for determining the effectiveness of a thiamine deficiency treatment, comprising determining the ratio of 4-methyl-5-(2-hydroxyethyl)-thiazole (TMA) to 2-methyl-4-amino-5-hydroxymethylpyrimidine (PMA) in a first biological sample taken from an animal before thiamine deficiency treatment and in a second biological sample taken from the animal after the thiamine deficiency treatment, wherein the thiamine deficiency treatment is effective if the ratio of TMA to PMA in the second sample is less than the ratio of TMA to PMA in the first sample.

16. (canceled)

17. (canceled)

18. (canceled)

19. (canceled)

20. (canceled)

21. The method of claim 15, wherein the ratios are determined by analyzing the samples by liquid chromatography/tandem mass spectrometry (LC-MS/MS).

22. The method of claim 15, wherein the second sample was taken from the animal from between about 10 hours to about 30 hours after thiamine deficiency treatment.

23. The method of claim 15, wherein the thiamine deficiency treatment comprises administering to the animal thiamine, a salt thereof, or a thiamine precursor.

24. (canceled)

25. The method of claim 23 wherein the thiamine salt is thiamine hydrochloride.

26. (canceled)

27. (canceled)

28. The method of claim 15, wherein the sample is urine.

29. (canceled)

30. (canceled)

31. The method of claim 15, wherein the animal is a human.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. A method to treat thiamine deficiency in an animal comprising administering thiamine, a salt thereof, or a thiamine precursor to the animal until the ratio of 4-methyl-5-(2-hydroxyethyl)-thiazole (TMA) to 2-methyl-4-amino-5-hydroxymethylpyrimidine (PMA) in a biological sample from the animal is less than five.

44. The method of claim 43, wherein the ratio is determined by analyzing the sample by liquid chromatography/tandem mass spectrometry (LC-MS/MS).

45. (canceled)

46. (canceled)

47. (canceled)

48. (canceled)

49. (canceled)

50. A method for treating thiamine deficiency in an animal in need of such therapy comprising: 1) administering thiamine, a salt thereof, or a thiamine precursor to the animal, 2) measuring the ratio of 4-methyl-5-(2-hydroxyethyl)-thiazole (TMA) to 2-methyl-4-amino-5-hydroxymethylpyrimidine (PMA) in a biological sample from the animal within 72 hours of administration, and 3) administering additional thiamine, a salt thereof, or a thiamine precursor to the animal if the ratio is greater than 5.

51. A method comprising treating thiamine deficiency in a human that is aware of having a ratio of 4-methyl-5-(2-hydroxyethyl)-thiazole (TMA) to 2-methyl-4-amino-5-hydroxymethylpyrimidine (PMA) of greater than 5 and that is therefore aware of being in need of such therapy comprising administering thiamine, a salt thereof, or a thiamine precursor to the human.

52. (canceled)
